(19) United States
(12) Patent Application Publication
(10) Pub. No.: US 2006/0286071 A1
(43) Pub. Date: Dec. 21, 2006

(54) Therapeutic pastes for medical device coating

(76) Inventors: Samuel J. Epstein, Watertown, MA (US); Wendy Naimark, Cambridge, MA (US)

Correspondence Address: KENYON & KENYON LLP, ONE BROADWAY, NEW YORK, NY, 10004, US

(21) Appl. No.: 11/158,452

(22) Filed: Jun. 21, 2005

(51) Int. Cl. A61K 48/00

(52) U.S. Cl. 424/93.2; 424/423; 514/44

(57) ABSTRACT

This invention provides a high-solids therapeutic composition for coating a medical device comprising: (a) a first material which is a hydrophilic therapeutic agent; and (b) a second material which includes a hydrophobic polymer and an emulsifying surfactant, wherein the composition is in a singular stable phase. Also provided is a mold-cast medical device, said medical device comprising a cured mixture of: (a) a first material which is a hydrophilic therapeutic agent; and (b) a second material which includes a hydrophobic polymer and an emulsifying surfactant, wherein the mixture forms a high-solids device. Further provided is an injectable polymer comprising: (a) a first material which is a hydrophilic therapeutic agent; and (b) a second material which includes a hydrophobic polymer and an emulsifying surfactant, wherein the polymer is in a singular stable phase.

### FIELD OF THE INVENTION

The present invention relates to therapeutic compositions for medical device coatings. More specifically, the present invention is directed to therapeutic pastes. The present invention also relates to devices which are cast in a mold and comprise a therapeutic composition. The present invention further relates to an injectable polymer scaffold comprising a therapeutic agent.

### BACKGROUND OF THE INVENTION

Medical devices are commonly coated with one or more therapeutic agents to facilitate delivery of the therapeutic agent(s) upon insertion or implantation of the device into the body. For example a stent or balloon catheter may be placed within an occluded blood vessel to prevent renarrowing, i.e. restenosis of the surrounding vessel, wherein the stent is coated with a composition comprising at least one anti-restenosis agent. A GDC® coil may be coated with thrombogenic fibers to aid in clot formation once the coil is placed inside the lumen of a brain aneurysm to occlude, i.e. fill, the aneurysm so as to prevent aneurysm rupture or re-rupture. Likewise, to prevent inflammation or rejection of an implanted device, an implanted device coating may comprise at least one anti-inflammatory agent. Therefore, coating a medical device with one or more therapeutic agents to deliver such agent(s) at or near its site of insertion or implantation diminishes adverse bodily reactions which may arise in response to the presence of the medical device and/or enhances the function of the implanted device. Medical device coatings may also be used to deliver therapeutic agents to augment treatment of an underlying disease, e.g., an angiogenic agent to induce formation of new blood vessels or nucleic acids encoding one or more proteins or growth factors required for treatment of a particular disease, e.g. cardiovascular disease.

The delivery of hydrophilic therapeutic agents from medical device coatings is problematic, however, since such agents are easily stripped away by contact with blood and bodily fluids during deployment of the device into the body. The amount of a therapeutic agent, e.g., DNA, which may come off the device during delivery of the device into the body varies depending upon the circumstances. A factor which influences the amount lost include the fact that DNA is readily soluble in aqueous media. This is particularly true in blood and other bodily fluids. During delivery of coated medical devices, blood contact with the coating is to be expected, as would be some dissolution of the DNA from the coating into the contacted blood or bodily fluid. Another influencing factor is that DNA is a brittle solid once it is dried. Thus, any manipulation of a device coated with just dried DNA would probably result in some of the DNA flaking off the device. In most cases, the release of the therapeutic agent requires incorporation of the agent into a polymer release platform. Since most of the polymers used for this purpose are hydrophobic, they are incompatible with hydrophilic solutions such as those comprising hydrophilic therapeutic agents. Although attempts to emulsify the aqueous agents in the polymer solution have been successful, the major drawback of the emulsified solutions is low loading of the therapeutic agent in the solid content of the emulsion, i.e., usually less than 1% of the therapeutic agent is incorporated into the resulting composition.

In many instances, the polymer release system is coated over the therapeutic layer. However, this technique is burdensome, as it requires two coating steps. In addition, there is little evidence to suggest that the release of the therapeutic agent may be controlled with a two layer coat.

### SUMMARY OF THE INVENTION

In one example embodiment of the present invention, a high-solids therapeutic composition for coating a medical device is provided which includes (a) at least two incompatible materials: (i) a first material which is a therapeutic agent; and (ii) a second material which includes a polymer; and (b) an emulsifying surfactant formulated with the with the at least two incompatible materials into a singular stable phase.

In another embodiment, a medical device having at least a portion thereof coated with the above-described therapeutic composition is provided.

In a further embodiment, a method of coating at least a portion of a medical device is provided, said method comprising: (a) providing a high-solids therapeutic composition for coating the medical device, said composition comprising: (i) a first material which is a therapeutic agent; and (ii) a second material which includes a polymer and an emulsifying surfactant, wherein the composition is in a singular stable phase; and (b) coating at least a portion of the medical device with the high-solids therapeutic composition.

In an example embodiment, a method of treating cardiovascular disease is provided, said method comprising: inserting into the heart muscle of a patient a medical device having at least a portion thereof coated with a composition comprising either a nucleic acid encoding an angiogenic factor or an angiogenic factor, a polymer and an emulsifying surfactant, wherein the composition is in a singular stable phase.

In another example embodiment, a method of treating atherosclerosis is provided, said method comprising: inserting into a blood vessel lumen of a patient a medical device having at least a portion thereof coated with a composition comprising (i) either a nucleic acid encoding an anti-restenosis agent, anti-inflammatory agent, a reverse cholesterol transport agent for plaque removal or a therapeutic agent itself, e.g., an anti-restenosis agent, anti-inflammatory agent, a reverse cholesterol transport agent for plaque removal; (ii) a polymer and (iii) an emulsifying surfactant, wherein the composition is in a singular stable phase.

In a further example embodiment, method of treating an intracranial aneurysm is provided, said method comprising: inserting into a brain aneurysm of a patient a medical device having at least a portion thereof coated with a composition comprising thrombogenic fibers, a polymer and an emulsifying surfactant, wherein the composition is in a singular stable phase.

Various drugs which may be delivered via the inventive compositions are discussed in the Detailed Description infra.

In another example embodiment of the present invention, a mold-cast medical device is provided, said medical device comprising a cured mixture of: (a) a first material which is a therapeutic agent; and (b) a second material which includes a polymer and an emulsifying surfactant, wherein the mixture forms a high-solids device. Examples of a mold-cast medical device include, but are not limited to, a film, a patch, a suture, a mesh, a plug, a tube and a clip.

In further example embodiment of the present invention, an injectable polymer is provided, said injectable polymer comprising: (a) a first material which is a therapeutic agent; and (b) a second material which includes a polymer and an emulsifying surfactant, wherein the polymer is in a singular stable phase.

Further aspects and advantages of the present invention will become apparent to those of ordinary skill in the art upon reading and understanding the following Detailed Description.

### BRIEF DESCRIPTION OF THE DRAWINGS

FIGS. 1A-1B illustrate the percentage of release of salmon sperm (ssDNA aq.) from a paste coating a stainless steel coupon, said paste comprised about 25-30% by weight of DNA, 65-70% by weight of SIBS[10] or SIBS[30] and 5-10% Poloxamer F127 (based on weight of solids), which occurred over time in minutes (FIG. 1A) and over time in days (FIG. 1B).

FIGS. 2A-2B demonstrate the phase separation (FIG. 2A) which occurred using a coating of a paste on a stainless steel coupon without poloxamer, the paste comprised 25% DNA and 75% SIBS; in contrast, FIG. 2B shows a stable emulsion formed with a paste coating comprising 25% DNA (based on weight of solids), 70% SIBS (based on weight of solids) and 5% Poloxamer F127. The coating comprising the poloxamer demonstrates stability, i.e., no phase separation.

### DETAILED DESCRIPTION OF THE INVENTION

High-solids homogeneous compositions comprising a relatively high content, preferably 10%-50%, of therapeutic agent compared to the total mass of solids are provided. The term “high-solids” as used herein refers to compositions having a solids content of from at least 50% up to about 100% of the total composition based on the total mass of solids in a composition and a very small percentage of solvent and/or water. In a preferred embodiment of the composition, the a paste emulsion comprises a high DNA:polymer ratio in which the total solids comprise from about 1% to about 100% of the total emulsion composition weight.

The compositions, preferably pastes, are created by combining a therapeutic agent, preferably a hydrophilic therapeutic agent, and a hydrophobic polymer mixed with an emulsifying surfactant. A composition may be comprised of as much as about 100% solids if the polymer added has a low concentration or as low as 1% solids if the polymer is a low molecular weight polymer. The term “paste” as used herein refers to a soft plastic (i.e., having the capacity for being molded or altered without breaking or tearing) mixture or composition which has a consistency that is stiffer than an ointment but has a higher percentage of solid ingredients, which make it less greasy than an ointment. The therapeutic hydrophilic agent may be in a viscous solution. Alternatively, the therapeutic hydrophilic may be atomized, microencapsulated or have a core/shell morphology.

The term “viscous” as used herein refers to a glutinous, i.e., glue-like, consistency and having a sticking or adhering quality. A “viscous solution” as used herein refers to a solution which has a glutinous consistency and adhering properties, and resists flow in a fluid or semifluid, i.e., a substance having both fluid and solid qualities. The term “atomized” as used herein refers to particles which are minute particles or a fine spray. The term “emulsifying surfactant” as used herein refers to any surface active material or wetting agent which allows two or more incompatible materials, e.g., a hydrophilic substance and a hydrophobic substance, to blend together so as to form a homogeneous mixture.

As used herein, a “singular stable phase” refers to a state in which two or more incompatible materials form a homogeneous mixture in which the two or more incompatible materials do not separate into two or more respective phases, i.e., no physically distinct states of the individual materials are apparent.

In one example embodiment, the hydrophobic polymer may be in viscous solution. In a further example embodiment, the hydrophobic polymer may be a liquid. In an alternative example embodiment, the hydrophobic polymer is not in solution. In another embodiment, the hydrophobic polymer may have a melting point at above room temperature, i.e., at about 75° F. Preferably, the emulsifying surfactant is added to the hydrophobic polymer and is mixed therewith prior to combining the mixture with the hydrophilic therapeutic agent. For example, a viscous solution of a therapeutic agent, e.g., DNA, may be added drop-wise to a material comprising a hydrophobic polymer and an emulsifying surfactant. Preferably, the material comprising the hydrophobic polymer and emulsifying surfactant is highly agitated while the therapeutic agent is added thereto. The therapeutic agent is added to the material comprising the hydrophobic polymer and emulsifying surfactant in a percentage that is suitable to form a stable paste. The percentage of therapeutic agent which is added will depend upon its molecular weight, i.e., an agent having a high molecular weight, e.g., 3-4 million g/mol will resist movement and stirring, therefore, a smaller percentage of such a therapeutic agent will be added in an amount sufficient to form a stable paste with the material comprising the hydrophobic polymer and emulsifying surfactant. The molecular weight of the hydrophobic polymer may also determine the concentration thereof which is added to the composition, e.g., a high molecular weight polymer will be added in a lower concentration so as to maintain a viscous solution. Preferably, the paste will comprise from about greater than 1% of a therapeutic agent by weight of solids in the composition after evaporation of water and/or solvents from the mixture.

Since both the therapeutic agent and polymer, e.g., a hydrophobic polymer, are incorporated into a single stable formulation, a medical device may be coated therewith in a single step. Additional layers of the provided singular stable phase compositions comprising other therapeutics may be added on top of this layer.

In an example embodiment of the present invention a high-solids therapeutic composition for coating a medical device is provided wherein the composition comprises at least two incompatible materials: (a) a first material which is a therapeutic agent; and (b) a second material which includes a polymer; and an emulsifying surfactant, wherein the composition is formulated in a singular stable phase. The therapeutic agent may be a hydrophilic therapeutic agent. The polymer may be a hydrophobic polymer.

Preferably, in the example embodiments described herein, the therapeutic agent may be in a viscous solution, atomized, microencapsulated, or have a core/shell morphology. In another preferred embodiment of the therapeutic composition, the ratio of therapeutic agent plus polymer:total mass of solids in the composition is greater than 1:100. In a still preferred embodiment, the ratio of therapeutic agent plus polymer:total mass of solids in the composition is from at least 25:100 up to 80:100. In a further example embodiment, the ratio of therapeutic agent:total mass of solids in the composition is greater than 1:100. More preferably, the ratio of therapeutic agent: total mass of solids in the composition is at least 25:100.

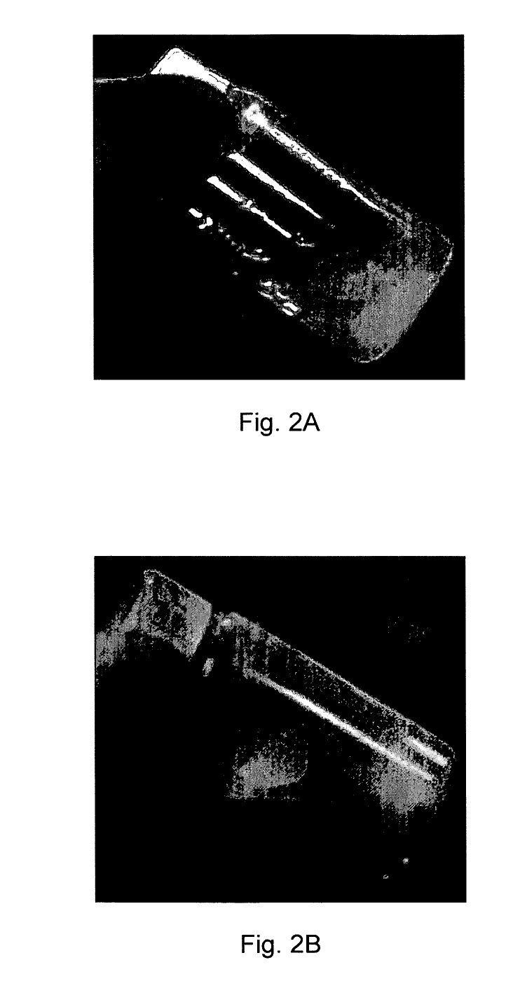

The inventive therapeutic compositions remain in a single stable phase, i.e., the components do not separate into separate and distinct incompatible non-homogeneous phases, e.g., hydrophilic and hyrophobic phases, throughout a coating process and are consistent and robust after the solvent and water evaporate from the coated medical device. FIG. 2B demonstrates the stable formulation achieved using a coating of paste containing 25% DNA and 70% SIBS (based on weight of solids) and 5% of the emulsifier F127 Poloxamer compared to the unstable coating of a paste preparation lacking F127 Poloxamer, in which phase separation occurred on the surface of the coated coupon, as shown in FIG. 2A.

In a preferred example embodiment, a ratio of therapeutic agent plus polymer:total mass of solids in the composition of greater than 1:100 is maintained after the therapeutic composition is coated on the medical device and dries thereon. In another example embodiment of the inventive compositions, the emulsifying surfactant is present as at least 5% up to 10% of the total mass of solids. In a preferred example embodiment, the emulsifying surfactant is F127 Poloxamer. Any emulsifying surfactant known to one of skill in the art may be used, including but not limited to an ionic surfactant, a lipid, and a detergent. Preferably the composition is a paste.

In an alternative embodiment, the composition is an injectable polymer comprising: (a) a first material which is a hydrophilic therapeutic agent; and (b) a second material which includes a hydrophobic polymer and an emulsifying surfactant, wherein the polymer is in a singular stable phase. The polymer creates a plug at an injection site to prevent back leaking of the therapeutic agent through the injection site. Once injected into a body site proximate to a medical device inserted into or implanted within the body, the polymer controls the in vivo release of the hydrophilic therapeutic agent. The injectable polymer of the present invention may be used to deliver at least one hydrophilic therapeutic to any of the devices discussed below. Preferably, the device is a Stiletto™ direct injection endomyocardial catheter. [ok?]

In a still further example embodiment, a device may be cast in a mold from the inventive compositions. The hydrophilic therapeutic agent may be combined with a material comprising a hydrophobic polymer and an emulsifying surfactant. The mixture may then be pressed into a mold, which is shaped in the form of a desired stand-alone device, e.g., a plug, a tube, a clip, a mesh, a film, a patch, a suture, and allowed to set in the mold by drying (curing), which removes any remaining solvent. The driving off of the solvent results in a high solids device. As discussed below, since the polymer is preferably a controlled release polymer, the therapeutic agent is released into the body once the device is inserted or implanted into a body part or lumen thereof, e.g., a blood vessel, since the implanted device swells on a molecular level, i.e., absorbs water, thereby providing a path for the hydrophilic therapeutic to be released from the device.

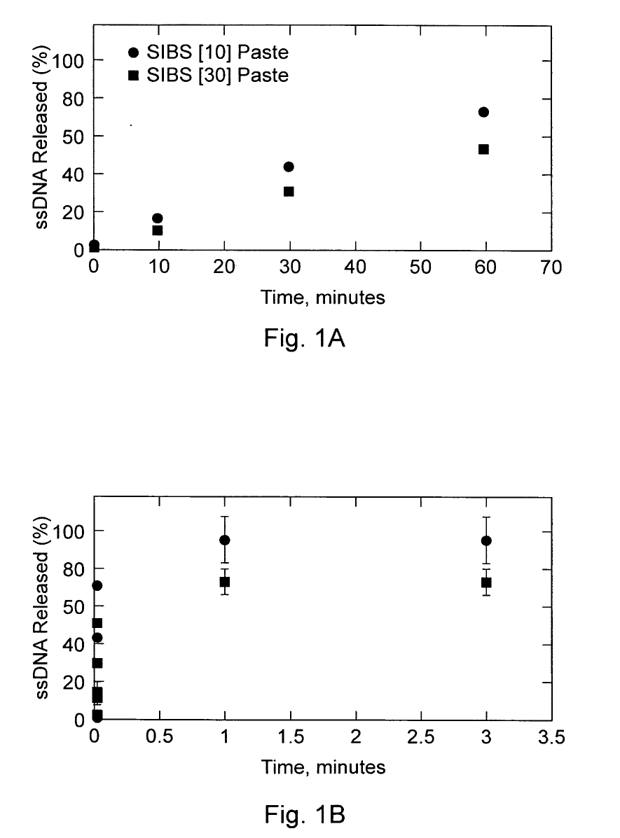

FIG. 1A illustrates the controlled release of a hydrophilic therapeutic agent, ssDNS, from a paste comprising ssDNA, F127 Poloxamer and SIBS[10], wherein almost 80% of the DNA was released over 60 minutes; a comparable, but slightly slower release of DNA was demonstrated from a paste comprising ssDNA, F127 Poloxamer and SIBS[30], which released almost 60% of the DNA during the same time period. Both pastes comprising ssDNA, F127 Poloxamer and either SIBS[10] or SIBS[30] provided a sustained release of DNA in 1 day, wherein the release did not differ statistically after 3 days, as shown in FIG. 1B, wherein the SIBS[10] paste released a higher percentage of ssDNA (almost 100%) at 1 day, respectively, compared to the nearly 80% DNA release attained from the SIBS[30] paste in the same time period, i.e., 1 day and 3 days respectively. The inventive compositions prevent release of the therapeutic agent, e.g., DNA, during delivery of the medical device into the body, i.e., provide slow release of therapeutic agent in an aqueous environment such that for the first 20-30 minutes, i.e., contact with blood and bodily fluids during deployment of the device into the body, less therapeutic agent is lost than from currently available polymer compositions. Once the coated device is inserted into the body, approximately 80%-100% of the therapeutic agent is released from the paste. In contrast, bare coronary stents coated with DNA under a laminate coating of 80% SIBS and 20% F127 Poloxamer release greater than 80% of the DNA in under 10 minutes. (data not shown)

Hydrophilic therapeutic agents may be delivered to a particular site in the body to treat a disease or disorder. In an example embodiment, cardiovascular disease may be treated by a method comprising: inserting into the heart muscle of a patient a medical device having at least a portion thereof coated with a composition comprising either a nucleic acid encoding an angiogenic factor or an angiogenic factor, a polymer and an emulsifying surfactant, wherein the composition is in a singular stable phase. The polymer may be a hydrophobic polymer. Preferably, the medical device is a catheter, more preferably a Stiletto™ direct injection endomyocardial catheter.

In another example embodiment, atherosclerosis may be treated by a method comprising: inserting into a blood vessel lumen of a patient a medical device having at least a portion thereof coated with a composition comprising either a nucleic acid encoding an anti-atherosclerosis agent or anti-atherosclerosis agent itself, a polymer and an emulsifying surfactant, wherein the composition is in a singular stable phase. Preferably, the medical device is a stent. The anti-atherosclerosis agent encoded by the nucleic acid may be an anti-restenosis agent. The polymer may be a hydrophobic polymer.

In a further example embodiment, an intracranial aneurysm may be treated by a method comprising: inserting into a brain aneurysm of a patient a medical device having at least a portion thereof coated with a composition comprising thrombogenic fibers, a hydrophobic polymer and an emulsifying surfactant, wherein the composition is in a singular stable phase. Preferably, the medical device is a GDC® coil.

In alternative embodiments of the methods of treatment provided, a composition comprising a therapeutic agent, a hydrophobic polymer and an emulsifying surfactant may be delivered to a site proximate to an inserted or implanted medical device by injection. For example, a composition comprising a therapeutic agent, such as an angiogenic factor, an anti-inflammatory agent, a vasodilator, or a beta blocker; a polymer; and an emulsifying surfactant may be injected proximate to a catheter, such as a Stiletto™ direct injection endomyocardial catheter, also called a Stiletto™ intramyocardial catheters. It is preferred that the injected composition is released near the needle, i.e., site of injection, to treat cardiovascular disease. The polymer may be a hydrophobic polymer.

In a further embodiment, a mold cast medical device comprising a hydrophilic therapeutic agent, as described above, may be used to deliver at least one particular hydrophilic therapeutic agent to a site proximate a disease or injury site from the medical device, e.g., an antibiotic may be delivered to a site of infection or surgical incision, or an antithrombogenic agent may be delivered to a blood vessel lumen or other tissue/organ at which undesired blood clots may form post-operatively, from a mold-cast tube or clip. Other drugs which may be delivered from the mold-cast devices, include but are not limited to, anti-infammatory agents, anti-restenotic agents, growth factors to enhance healing. Additional drugs which also may be delivered using the compositions and devices provided are described infra.

The term “therapeutic agent” as used herein includes one or more “therapeutic agents” or “drugs”. The terms “therapeutic agents” and “drugs” are used interchangeably herein.

The therapeutic agent may be any pharmaceutically acceptable agent such as a non-genetic therapeutic agent, a biomolecule, a small molecule, or cells. The therapeutic agent which may be used in the compositions, e.g., paste and devices provided herein may be hydrophilic, however, even in aqueous solutions, some hydrophobic therapeutic agents may also be formulated into the compositions provided herein depending upon the surfactant used. Therefore, the compositions are not limited to the use of hydrophilic therapeutic agents.

Exemplary non-genetic therapeutic agents include anti-thrombogenic agents such heparin, heparin derivatives, pro staglandin (including micellar pro staglandin El), urokinase, and PPack (dextrophenylalanine proline arginine chloromethylketone); anti-proliferative agents such as enoxaprin, angiopeptin, sirolimus (rapanycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; angiotensin converting enzyme (ACE) inhibitors; beta-blockers; congestive heart failure drugs; anti-arrhythmic drugs; bAR kinase (bARKct) inhibitors; phospholamban inhibitors; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include serca-2 protein, monocyte chemoattractant proteins (“MCP-1”) and bone morphogenic proteins (“BMPs”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNAs encoding them. Non-limiting examples of genes include survival genes that protect against cell death, such as anti-apoptotic Bcl-2 family factors and Akt kinase; serca-2 gene; and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered. Non-limiting examples of cells include side population (SP) cells, lineage negative (Lin⁻) cells including Lin⁻CD34⁺, Lin⁻CD34^{+, Lin}⁻cKit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, go cells, endothelial cells, adult cardiomyocytes, fibroblasts, smooth muscle cells, adult cardiac fibroblasts +5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable.

Non-limiting examples of suitable non-biodegradable polymers include polystyrene; polyisobutylene copolymers and styrene-isobutylene block copolymers such as styrene-isobutylene-styrene tri-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL)®; squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized, e.g., atomized, particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. One of skill may vary the composition layers, e.g., the first layer may be a tie layer. [ok?] Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters (e.g., a Stiletto™ intramyocardial delivery catheter), guide wires, balloons, filters (e.g., vena cava filters), stents, stent grafts, vascular grafts, intraluminal paving systems, implants, patches, slings, meshes, sutures, films, and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

The invention will be better understood from the examples which follow, however the invention is not limited to these examples, which are solely intended to be illustrative thereof.

### EXAMPLES

Modulating DNA Release from Novel DNA/SIBS Pastes

Pastes were formulated into a DNA/SIBS emulsion for coating a medical device with salmon sperm 2% DNAaq. (ssDNA) [Sigma-Aldrich], 15% SIBS[30] (30% styrene) or 15% SIBS[10] (10% styrene), and 5% F127 Poloxamer [BASF] in toluene as follows. Other solvents which may be used in these compositions, include but are not limited to, halogenated solvents, e.g., used to maintain high lipophilic phase density, THF, MIBK, benzene, and other solvents known to one of skill in the art.

TABLE 1
Final Paste Formulation:

| Paste Component | Mass Percent of Total Solids |
|---|---|
| DNA | 25%-30% |
| SIBS | 65%-70% |
| F127 | 5%-1O% |

TABLE 2
Coating Matrix:

| | |
|---|---|
| SIBS[30] | SIBS[10] |

| Coating | DNA | Coating | DNA |
|---|---|---|---|
| Mass (μg) | Mass (μg) | Mass (μg) | Mass (μg) |
| 890 | 265 (29.8%) | 618 | 151 (24.4%) |
| 875 | 261 (29.8%) | 684 | 167 (24.4%) |
| 841 | 250 (29.7%) | 696 | 170 (24.4%) |

To determine the changes which occur in the release profile of DNA from the stable DNA/SIBS coating, the amount of emulsifying Poloxamer was varied and the pastes were coated onto a stainless steel coupon.

As shown in FIG. 1A, the paste comprising approximately 25-30% ssDNA, 5-10% F127 Poloxamer and 65-70% SIBS[10] (by weight of the total solids) provided a steady release of ssDNA of nearly 80% over 60 minutes, while the paste comprising approximately 25-30% ssDNA, 5-10% F127 Poloxamer and 65-70% SIBS[30] released almost 60% during the same time period. The release for both was an in vitro release. Importantly, less than 20% of the DNA was released at about 10 minutes when using either composition. Therefore, a stent coated with such a composition comprising DNA, polymer and an emulsifying surfactant such as a poloxamer may be delivered into a blood vessel with minimal loss of DNA, thereby providing increased efficacy.

The release profile of ssDNA from a paste comprising either approximately 25-30% ssDNA, 5-10% F127 Poloxamer and 65-70% SIBS[10] or SIBS[30] is shown in FIG. 1B. Both pastes provided an in vitro release of approximately 80% to 100% of the therapeutic agent within 1 day with no statistically significant increase in additional release by the end of 3 days, wherein the SIBS[10] paste released a higher percentage of ssDNA (approximately 100%) within 1 day, respectively, compared to the approximately 80% release obtained from the SIBS[30] paste within one day.

Although the invention has been described with reference to the preferred embodiments, it will be apparent to one skilled in the art that variations and modifications are contemplated within the spirit and scope of the invention. The drawings and description of the preferred embodiments are made by way of example rather than to limit the scope of the invention, and it is intended to cover within the spirit and scope of the invention all such modifications and alterations insofar as they come within the scope of the appended claims or the equivalence thereof. All documents and publications cited herein are expressly incorporated by reference in their entireties into the subject application.

What is claimed is:

1. A high-solids therapeutic composition for coating a medical device comprising: (a) at least two incompatible materials:
(i) a first material which is a therapeutic agent; and
(ii) a second material which includes a polymer; and (b) an emulsifying surfactant formulated with the at least two incompatible materials into a singular stable phase.

2. The therapeutic composition of claim 1, wherein the ratio of therapeutic agent plus polymer:total mass of solids in the composition is greater than 1:100.

3. The therapeutic composition of claim 2, wherein the ratio of therapeutic agent plus polymer:total mass of solids in the composition is from at least 25:100 up to 60:100.

4. The therapeutic composition of claim 1, wherein the ratio of therapeutic agent: total mass of solids in the composition is greater than 1:100.

5. The therapeutic composition of claim 4, wherein the ratio of therapeutic agent: total mass of solids in the composition is at least 25:100.

6. The therapeutic composition of claim 1, wherein a ratio of therapeutic agent plus polymer:total mass of solids in the composition of greater than 1:100 is maintained after the therapeutic composition is coated on the medical device and dries thereon.

7. The therapeutic composition of claim 1, wherein the emulsifying surfactant is present as about 1.0 to 20% of the total mass of solids.

8. The therapeutic composition of claim 7, wherein the emulsifying surfactant is a di-block co-polymer of polyoxyethylene and polyoxypropylene, a lipid or a detergent.

9. The therapeutic composition of claim 1, wherein the polymer is a biodegradable polymer, a non-biodegradable polymer, or a combination thereof.

10. The therapeutic composition of claim 1, wherein the therapeutic agent is a nucleic acid, a protein, peptide or a small molecule drug.

11. The therapeutic composition of claim 12, wherein the nucleic acid is DNA or RNA.

12. The therapeutic composition of claim 13, wherein the DNA is naked DNA or is incorporated into a viral vector.

13. The therapeutic composition of claim 1, wherein the device is a stent, a catheter, a guide wire, a balloon, a filter, a stent graft, a vascular graft, an intraluminal paving system, an implant, a film, a suture, a patch, a mesh or a sling.

14. A medical device having at least of portion thereof coated with the therapeutic composition of claim 1.

15. The medical device of claim 16, which is a stent, a catheter, a guide wire, a balloon, a filter, a stent graft, a vascular graft, an intraluminal paving system, an implant, a film, a suture, a patch, a mesh or a sling.

16. A mold-cast medical device, said medical device comprising a cured mixture of: (a) at least two incompatible materials:
(i) a first material which is a therapeutic agent; and
(ii) a second material which includes a polymer; and (b) and an emulsifying surfactant, wherein the mixture forms a high-solids device.

17. The medical device of claim 16 wherein the ratio of therapeutic agent plus polymer:total mass of solids in the composition is greater than 1:100.

18. The medical device of claim 16, wherein the ratio of therapeutic agent plus polymer:total mass of solids in the composition is from at least 25:100 up to 60:100.

19. The medical device of claim 16, wherein a ratio of therapeutic agent plus polymer:total mass of solids in the composition of greater than 1:100 is maintained after the device is inserted into a body.

20. The medical device of claim 16, wherein the device remains as a singular stable high-solid upon insertion into a body.

21. The medical device of claim 16, wherein the emulsifying surfactant is present as at least 10% of the total solid mass of the high-solid.

22. The medical device of claim 21, wherein the emulsifying surfactant is a di-block co-polymer of polyoxyethylene and polyoxypropylene, a lipid or a detergent.

23. The medical device of claim 16, wherein the polymer is a biodegradable polymer, a non-biodegradable polymer, or a combination thereof.

24. The medical device of claim 16, wherein the therapeutic agent is a nucleic acid, a protein or a small molecule drug.

25. The medical device of claim 24, wherein the nucleic acid is DNA or RNA.

26. The medical device of claim 35, wherein the DNA is naked DNA or is placed in a viral vector.

27. The medical device of claim 26, which is a plug, a tube, a clip, a patch, a film, a suture, a patch, a mesh or a sling.
